(19) United States
(12) Patent Application Publication
(10) Pub. No.: US 2023/0270972 A1
(43) Pub. Date: Aug. 31, 2023

(54) HUMAN SIMULATOR

(71) Applicants: Martha Hall, Landenberg, PA (US); Gabriella Zarlenga, Newark, DE (US)

(72) Inventors: Martha Hall, Landenberg, PA (US); Gabriella Zarlenga, Newark, DE (US)

(73) Assignee: University of Delaware, Newark, DE (US)

(21) Appl. No.: 18/143,307

(22) Filed: May 4, 2023

Related U.S. Application Data
(63) Continuation of application No. PCT/US2021/058183, filed on Nov. 5, 2021.
(60) Provisional application No. 63/109,884, filed on Nov. 5, 2020.

(51) Int. Cl. A61M 21/02

(52) U.S. Cl. CPC A61M 21/02; A61M 2021/0022; A61M 2240/00

(57) ABSTRACT

A monitoring system for use with a human simulator for simulating interaction between a human baby and a bonded human and method of operating thereof are disclosed. The simulator comprises a housing having an outer layer configured to contact the baby, the housing having a surface contoured to simulate a torso of the bonded human; a respiration simulation system configured to simulate a respiration motion of the bonded human; and a heartbeat simulation system configured to simulate a heartbeat of the bonded human. The monitoring system comprises a smart device having a first communication interface; one or more sensors for detecting one or more biological parameters of the bonded human; at least one processor programmed with machine readable instructions to activate the sensor(s) and to transmit a signal corresponding to the detected biological parameters from the first communication interface to a second communication interface connected to the simulator.

### CROSS REFERENCE TO RELATED APPLICATIONS

This application claims priority from U.S. Provisional Application Ser. No. 63/109,884, titled HUMAN SIMULATOR FOR NEONATAL CARE, filed Nov. 5, 2020, incorporated herein by reference in its entirety.

### FIELD OF THE INVENTION

The present invention relates generally to medical simulations, and more particularly, to simulation devices for use in neonatal care and management.

### BACKGROUND OF THE INVENTION

Maintaining a midline head alignment for human babies born premature and/or requiring medical care can help optimize musculoskeletal development and decrease the risk of neurodevelopmental complications. Keeping the baby in a stationary position with this midline head alignment is critical, especially in the first few weeks of care. In addition, Kangaroo care, more commonly known as skin-to-skin contact (SSC) between the baby and another human (e.g. the mother or a medical professional) is also important, but such care may require moving the baby, e.g. in and out of the incubator. Such movement can undesirably impact the midline head alignment of the baby. Accordingly, improved systems and devices are desired for improving premature infant health outcomes by simultaneously providing the benefits of midline head alignment and Kangaroo care.

### SUMMARY OF THE INVENTION

Aspects of the present invention are directed to simulation systems and devices for use in neonatal care.

In accordance with one aspect of the present invention, a human simulator configured to simulate interaction between a human baby and a bonded human is disclosed. The human simulator comprises a housing having an outer layer configured to contact the baby, the housing having a surface contoured to simulate a torso of the bonded human; a respiration simulation system positioned at least in part within the housing and configured to simulate a respiration motion of the bonded human; and a heartbeat simulation system positioned at least in part within the housing and configured to simulate a heartbeat of the bonded human.

In accordance with another aspect of the present invention, a method for using a human simulator configured to simulate interaction between a human baby and a bonded human is disclosed. The method comprises positioning the human baby on an outer layer of the human simulator; activating a respiration simulation system to simulate a respiration motion of the bonded human; and activating a heartbeat simulation system to simulate a heartbeat of the bonded human.

In accordance with yet another aspect of the present invention, a monitoring system for use with a human simulator configured to simulate interaction between a human baby and a bonded human is disclosed. The monitoring system comprises a smart device having a first communication interface; one or more sensors configured to detect one or more biological parameters of the bonded human; at least one processor; wherein the at least one processor is programmed with machine readable instructions to activate the one or more sensors and to transmit a signal corresponding to the detected biological parameters of the bonded human from the first communication interface via a global communication network to a second communication interface connected to the human simulator.

In accordance with another aspect of the present invention, a non-transitory computer memory medium programmed with machine readable instructions for operating a human simulator configured to simulate interaction between a human baby and a bonded human is disclosed. The human simulator comprises a heartbeat simulation system configured to simulate a heart rate of a bonded human and a respiration simulation system configured to simulate a respiration rate of the bonded human. The machine readable instructions comprises instructions for causing the heartbeat simulation system and the respiration simulation system to be activated in accordance with an algorithm or program stored in the computer memory medium.

### BRIEF DESCRIPTION OF THE DRAWINGS

The invention is best understood from the following detailed description when read in connection with the accompanying drawings, with like elements having the same reference numerals. When a plurality of similar elements are present, a single reference numeral may be assigned to the plurality of similar elements with a small letter designation referring to specific elements. When referring to the elements collectively or to a non-specific one or more of the elements, the small letter designation may be dropped. This emphasizes that according to common practice, the various features of the drawings are not drawn to scale unless otherwise indicated. On the contrary, the dimensions of the various features may be expanded or reduced for clarity. Included in the drawings are the following figures:

FIGS. 1A-1B depict an exemplary human simulator in accordance with aspects of the present invention;

FIG. 2 depicts another exemplary human simulator;

FIGS. 3A-3B depicts an exemplary outer layer of the human simulator;

FIG. 4 depicts a cross-section view of the human simulator, showing an exemplary heartbeat simulation system and an exemplary respiration simulation system;

FIGS. 5A-5B and 6A-6B depict details of the respiration simulation system;

FIG. 7 depicts an exemplary monitoring system for use with the human simulator;

FIG. 8 depicts another embodiment of the human simulator; and

FIG. 9 depicts an exemplary method of using the human simulator.

### DETAILED DESCRIPTION OF THE INVENTION

Aspects of the invention are described herein with reference to simulating specific neonatal care and management of human babies, who may be born premature and/or requiring medical care. It will be understood by one of ordinary skill in the art that the exemplary devices described herein are not limited to a specific type of care and management or to specific patients requiring medical care. Other types of medical care and treatment suitable for use with the disclosed devices will be known to one of ordinary skill in the art from the description herein.

It will be appreciated that throughout this specification the term “bonded human” is to be broadly construed to include a provider of neonatal care, management or treatment. A bonded human may also include family members who are naturally and/or legally related to the infant patient (e.g. mother, father, etc.). The term may also include trainees and professionals in the field of medicine, particularly in the fields of obstetrics, midwifery, nursing, and emergency medical services, as well as non-health care professionals. Further, mentions of organs and other segments of human anatomy are intended to refer to anatomical models simulating or emulating functions or responses of their biological counterparts. The bonded human may be a singular individual, a simulated individual (e.g. from information generated by a computer or selected to represent a combination of individuals), or may represent more than one individual over different periods of time (e.g. the neonatal care professional on duty that changes by shift, both biological or adoptive parents, and the like).

Finally, the term “neonatal care” may comprise performing supportive positioning on the baby, such as placing infants or babies in a resting position that is conducive to promoting proper musculoskeletal development and/or reducing the risk for neurodevelopmental complications. A non-limiting example of such supportive positioning is placing the baby in a head midline position. Neonatal care may also include Kangaroo care, a method of holding a baby involving direct skin-to-skin contact. Kangaroo care can be used to promote good infant health outcomes, i.e., help the baby’s recovery and/or development, by having direct skin to skin contact between the baby and the bonded human. More specifically, the body temperature, the beating heart and respiration of the bonded human can maintain or enhance the baby’s progress towards recovery and/or development.

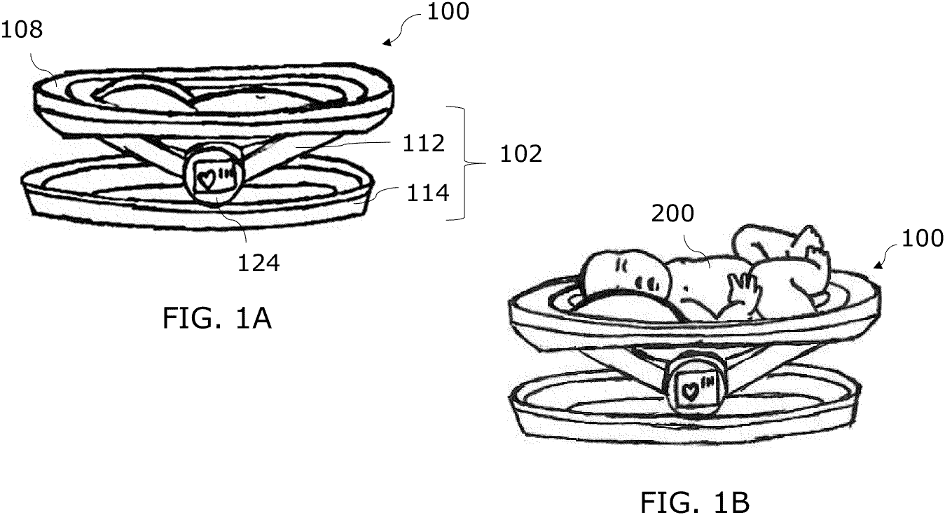

With reference to the drawings, FIGS. 1A-1B illustrates an example human simulator 100 in accordance with aspects of the present invention. Simulator 100 is usable for simultaneously providing the benefits of midline head alignment and skin-to-skin contact. The simulator 100 can be adjusted to provide neonatal care in various environments (e.g. hospital or other sterile settings, trauma or critical care sites, residential home, etc.).

In general, human simulator 100 is configured to simulate interaction between a human baby 200 and a bonded human 202. Simulator 100 can include a housing 102, a respiration simulation system 104, and a heartbeat simulation system 106. Additional details of simulator 100 are described below.

### Housing

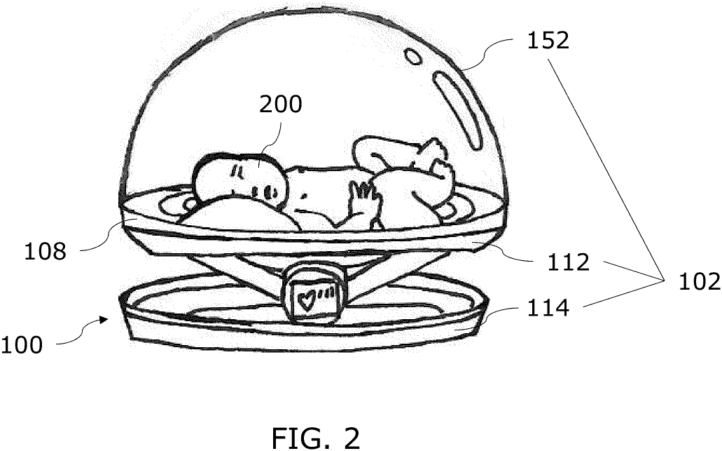

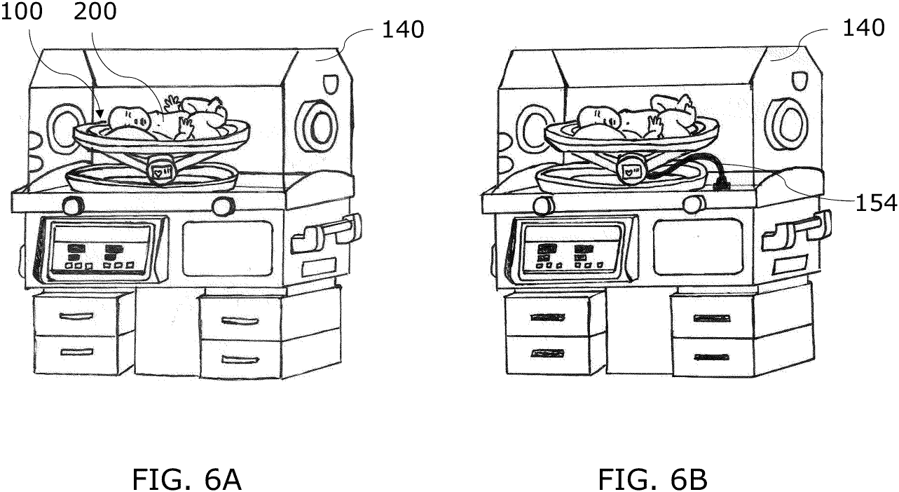

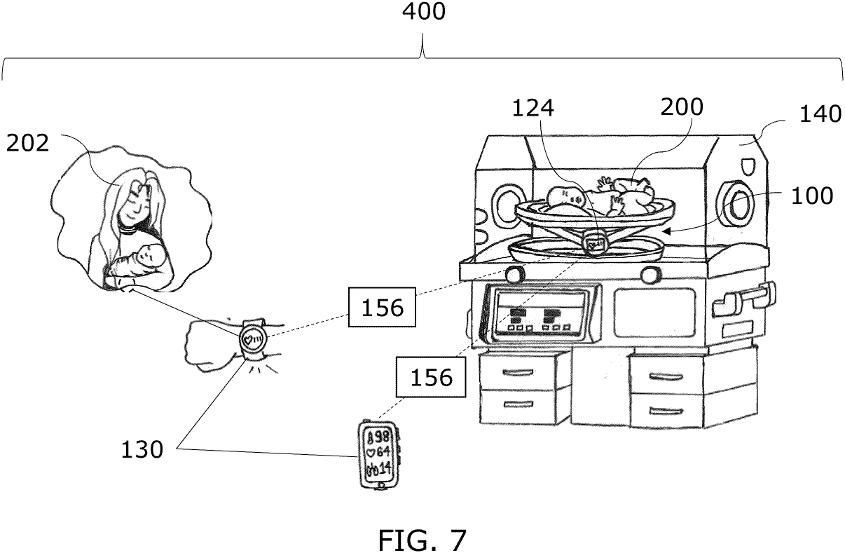

Housing 102 houses operational components which simulate and/or provide neonatal care. Such components comprise the respiration simulation system 104 and the heartbeat simulation system 106. In an example, as seen in FIGS. 1A-1B, the housing 102 has a size and shape that is intended to simulate a torso of the bonded human 202 (FIG. 7). Additionally or optionally, the housing 102 may have a size and shape that corresponds to a specific medical environment, such as the interior space of an incubator 140 (FIGS. 6A-6B). Further, the housing 102 may include cover 152 (FIG. 2) which is configured to be attached to outer layer 108, such that outer layer 108 and cover 152 create an interior within which baby 200 is positioned. Cover 152 may comprise glass (or transparent or semi-transparent plastic, such as but not limited to polycarbonate) to facilitate monitoring of the baby 200. In some examples, housing 102 incorporates or may be connected to a number of separate components, which are included in the prescribed care and management of the infant patient, such as human baby 200.

The housing 102 can be formed from one or more structures which together define a cavity or space 110, in which the operational components of the simulator 100 (e.g. the respiration simulation system 104, the heartbeat simulation system 106, etc.) are provided or positioned, thereby providing protection for at least these components and concealing wiring and other items. Additional details of these operational components are discussed further below.

In an exemplary embodiment shown in FIGS. 1A-1B, the housing 102 comprises a base 114 and a support frame 112 that extends upwardly from the base 114 and that is generally Y-shaped from the side view as depicted. Although the housing 102 is illustrated as being integrally formed as a single body of unitary construction, one of ordinary skill in the art would understand from the description herein that the housing 102 may be comprised of separate components, e.g. support frame 112 and base 114. The base 114 is illustrated in FIGS. 1A-1B as having a rectangular geometry with rounded corners, but it should be understood from the description herein that the base 114 may have alternative geometries. Likewise, the support frame 112 may have an alternative shape from the Y-shape depicted in FIGS. 1A-1B. For example, in alternative embodiments, the housing 102 may be a closed system having side panels around an entire periphery of the housing 102 (e.g. a box shape). In this configuration, housing 102 may be mounted on a frame configured to support at least the housing 102, such that the housing is elevated relative to a base of the frame.

In an exemplary embodiment, the support frame 112 and the base 114 are each made of relatively durable or rigid materials (i.e. relatively more durable and rigid than the components in contact with the baby). Relatively durable or rigid materials may include but are not limited to ABS plastic, Polycarbonate (PC), PC/ABS mixture, High Density Poly Ethylene (HDPE), nylon, acetal, composites (e.g. carbon fiber reinforced), or a combination thereof. Still further, relatively durable or rigid materials may include metal, such as stainless steel (e.g. 316L, 303, or 304 alloys) and anodized aluminum, or alloys thereof. Relatively durable or rigid materials may be a combination of plastic and metal, as stated above. The relatively rigid material is intended to provide support and/or a mounting structure for the components of the simulator 100. The support frame 112 and the base 114 may be made of the same materials, but it would be understood from the description herein that optionally, the support frame 112 and base 114 may comprise different materials. The surface of the housing 102 that is contoured to simulate the torso of the bonded human 202 may be provided by the support frame 112. Additionally or optionally, the surface of the housing 102 that is contoured to simulate the torso the bonded human 202 is provided by an outer layer 108 configured to be attached to the support frame 112. Attachment of outer layer 108 to support frame 112 may be performed via adhesives and/or other known attachment mechanisms, such as fasteners, microloop/microhook (e.g. Velcro® brand)-type materials, mechanical interface locking mechanisms (e.g. a snap fit between one component of simulator 100 and another component of simulator 100, such as between outer layer 108 and support frame 112). However, outer layer 108 and support frame 112 may be integrally formed as a single body of unitary construction.

The outer layer 108 of housing 102 is configured to contact the baby 200. In an exemplary embodiment, the outer layer 108 may extend partially or entirely over and about the support frame 112, such that the outer layer 108 conforms to the surface of the support frame 112. Thus, the exterior surface of the support frame 112 may simulate the torso of the bonded human 202. Additionally or optionally, the outer layer 108 or a portion thereof may be independently contoured to simulate the torso of the bonded human 202, including a bespoke contour parametrically matched to the unique details of a specific bonded human 202 (e.g. the baby’s mother). Further, the outer layer 108 of the support frame 112 comprises material intended to mimic the human skin. In particular, the outer layer 108 comprises material having one or more properties (e.g. the look and feel) selected to simulate human skin. This material may comprise an elastic material such as silicone. Other suitable materials for use in simulating human skin will be generally known to one of ordinary skill in the art from the description herein. In addition, the material of outer layer 108 may be customizable in coloring and texturing to match a variety of skin tones. That the material of outer layer 108 is intended to mimic or simulate one or more properties of human skin is desirable for simulating direct skin to skin contact between the baby 200 and the bonded human 202, as would be the case in standard Kangaroo care.

Still further, the outer layer 108 and the support frame 112 are useful to conceal components of simulator 100, such as the respiration simulation system 104 and the heartbeat simulation system 106, thereby protecting the operational components of simulator 100 from contamination or possible interference by other equipment that may be within the vicinity of the simulator 100 (e.g. hospital equipment, medical devices, etc.). Importantly, to maintain a sterile and safe outer layer 108, which is configured to contact the baby 200, the outer layer 108 may comprise anti-microbial (e.g. anti-bacterial, anti-fungal, anti-viral, anti-parasitic, or other biocidal) material. In one exemplary embodiment, a surface coating may be applied to the outer layer 108, such that bacterial growth is mitigated or prevented, without the need for chemical disinfectants, which may be of decreasing effectiveness over time (due to increasing microbe resistance) and/or may be harmful for the baby 200. In an exemplary embodiment, the anti-microbial material may comprise anti-microbial surface microstructures, such as are present in materials designed and manufactured by Sharklet Technologies, Inc. of Aurora, Colorado, United States, which microstructures may be embodied in the at least the outer layer 108 of the simulator 100. In another exemplary embodiment, the anti-microbial material may comprise anti-microbial additive, such as GERM ARMOR®, as designed and manufactured by POLYMORE MATERIALS of Singapore, which antimicrobial additive may be embodied in the at least the outer layer 108 of the simulator 100. In another exemplary embodiment, the anti-microbial material may comprise surface coatings, such as microRESIST® Antimicrobial Parylene Technology, as designed and manufactured by Specialty Coating Systems of Indianapolis, Indiana, which surface coating may be embodied in the at least the outer layer 108 of the simulator 100. In another exemplary embodiment, the anti-microbial material may comprise anti-viral and/or anti-bacterial paint, such as Copper Armor™, as designed and manufactured by PPG Industries, Inc. of Pittsburgh, Pennsylvania, which paint may be embodied in the at least the outer layer 108 of the simulator 100. It should be understood that the anti-microbial material may further comprise a combination of two or more anti-microbial materials discussed above, and is not limited to any particular anti-microbial material(s) or combinations thereof. In another exemplary embodiment, the anti-microbial material may comprise a removeable and/or disposable anti-bacterial protective sheet that can be releasably attached to the exterior surface of at least the outer layer 108. One or more anti-bacterial protective sheets may be attached to the outer layer 108 at the beginning of each use cycle of the simulator 100 and/or may be replaced at regular intervals throughout the use cycle of the simulator 100.

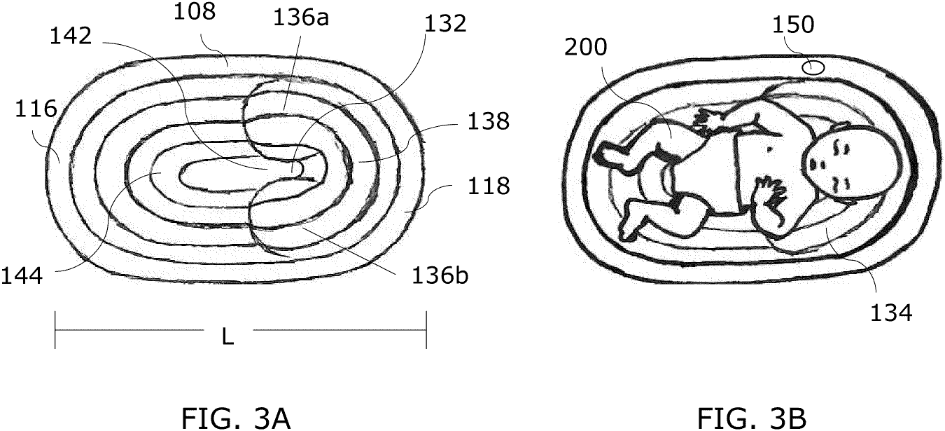

In an exemplary embodiment, as shown in FIGS. 3A-3B, the housing 102 extends along a length (L) between a first end portion 116 and a second end portion 118. One or both of the first end portion 116 and second end portion 118 may include a concave area 132 for receiving a head portion of the human baby. The degree of concavity of the concave area 132 may be selected for supporting the head of the baby 200 when the baby 200 is resting in a specific body position (e.g. supine position, prone position, or lateral position, etc.). In one non-limiting example, the concave area 132 may have 5-45 degrees of concavity for supporting the head of the baby 200 in a spinally neutral position when the baby is resting in a lateral position (i.e. on its side). In another non-limiting example, the concave area 132 may be selected for allowing medical equipment, such as endotracheal tube holders, to be comfortably positioned in contact with the baby 200, such as when the baby 200 is placed in a prone position.

Adjacent the concave area 132 may include raised surfaces having one or more properties (e.g. size, shape, contours) that may be selected in accordance with the head midline alignment position. In one non-limiting example, the raised surfaces are configured to contact the head and neck of baby 200 for encouraging the proper head midline position. The raised surfaces or contours may provide supportive positioning by providing gentle resistance, but do not completely restrict the baby’s movement. In an exemplary embodiment, as shown in FIGS. 3A-3B, the raised surfaces may comprise two arms 136 and a back section 138 integrally formed into a generally “U-shaped” or “C-shaped” pillow 134. The arms 136 and back section 138 together create an interior opening 142, which roughly extends around the concave area 132 of the outer layer 108, such that the pillow 134 may easily and comfortably encircle the head, neck, and/or shoulder portions of the baby 200. The pillow 134 may be configured to provide supportive positioning for the baby 200, such as encouraging the proper head midline position. The pillow 134 may comprise different material relative to outer layer 108. In an exemplary embodiment, pillow 134 may comprise softer material (relative to other components of simulator 100, such as housing 102), such that the pillow 134 can provide gentle or comfortable support to the head and neck portions of the baby 200, and yet is firm enough to guide the head and neck portions of the baby 200 toward appropriate head midline alignment.

In one non-limiting example, pillow 134 may comprise an outer layer of natural or synthetic flexible elastomer filled with air, fluid, gel, or foam or other compressible or granular solid, or a combination thereof. Further, to maintain a sterile and safe pillow 134, which is configured to contact the baby 200, the pillow 134 may comprise anti-microbial (e.g. anti-bacterial, anti-fungal, anti-viral, anti-parasitic, or other biocidal) material. In an exemplary embodiment, pillow 134 comprises similar anti-microbial material as discussed above with respect to outer layer 108.

Although FIGS. 2A-2B show that pillow 134 may be positioned in one end portion (e.g. second end portion 118), it should be understood that pillow 134 may be positioned in both end portions 116, 118. When both of the first end portion 116 and second end portion 118 include the concave area 132 and pillow 134, and the contours of the outer layer 108 are symmetrical when comparing the first end portion 116 and the second end portion 118, then the baby 200 may be positioned on either end portion 116/118 of simulator 100. Further, although FIGS. 2A-2B show that pillow 134 and outer layer 108 are integrally formed as a unitary structure, it should be understood that pillow 134 may be a distinct structure that is separate and removeable from outer layer 108 and/or housing 102. Additionally or optionally, the pillow 134 may be adjustable in position relative to outer layer 108, for accommodating different sizes of baby 200, or for accommodating other aspects of the environment in which simulator 100 is used (e.g. incubator 140, etc.).

In another exemplary embodiment, the outer layer 108 may have a surface topography configured to uniformly or evenly distribute contact forces between the outer layer 108 and the baby 200. In this configuration, relatively high-pressure (i.e. relative to other points of contact across outer layer 108) points of contacts between the outer layer 108 and the baby 200 is reduced. Accordingly, the outer layer 108 may reduce or prevent the risk/likelihood of developing skin ulcers and/or developing head deformations, such as plagiocephaly (flat head syndrome). Additionally or optionally, the surface topography of outer layer 108 may permit easier breathing when the baby 200 is resting in a prone or supine position and/or the baby 200 is intubated.

Still further, the outer layer 108 may have a surface topography that is moldable to accommodate more than one size and weight of the baby 200. Additionally or optionally, the surface topography of outer layer 108 may be moldable to provide supportive positioning, i.e. guide the baby 200 to rest in a proper head midline position.

### Respiration Simulation System

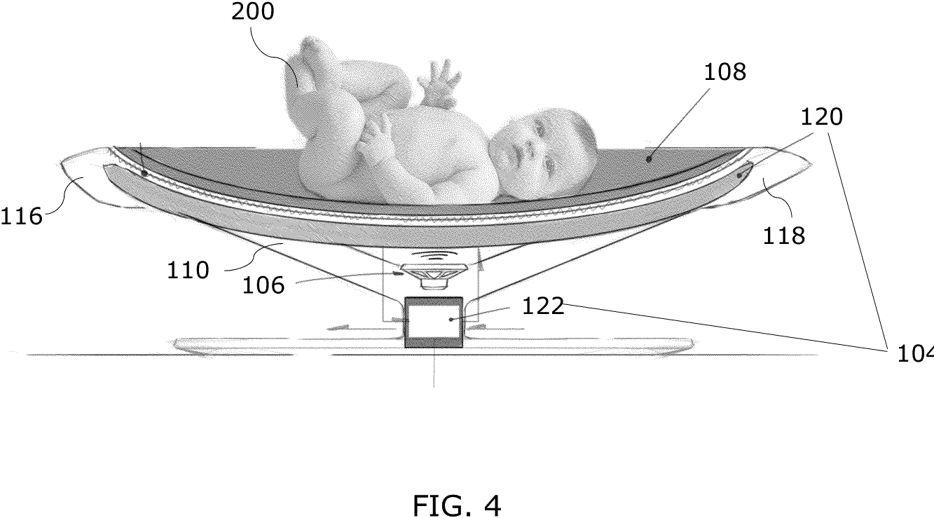

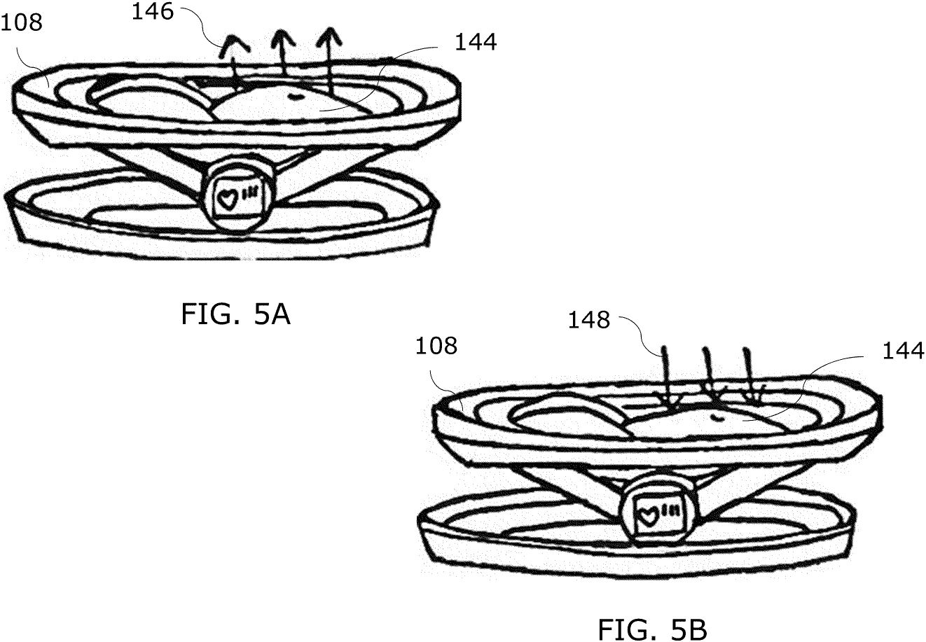

Positioned within the housing 102, the respiration simulation system 104 is configured to simulate a respiration motion of the bonded human 202. As shown in FIGS. 4 and 5A-5B, the respiration simulation system 104 comprises a bladder system comprising an inflatable bladder 120 having a relatively inflated state (as shown in FIG. 5A) and a relatively deflated state (as shown in FIG. 5B). It should be understood that a bladder may have a continuum of states between fully deflated (0% inflated) and fully inflated (100% inflated) and therefore the term “relatively inflated state” as used herein means a state that is more inflated (e.g. has a higher inflation percentage) than a “relatively deflated state” as that term is used herein. Thus, both the relatively inflated state and relatively deflated state may lie anywhere on the inflation continuum of the bladder 120 and may not refer to any specific inflation percentage; what is important is that the movement from relatively inflated to relatively deflated entails a movement between a greater inflation percentage and a lesser inflation percentage, as expressed as a percentage of a fully inflated bladder 120. The range or span of the inflation percentages may be fixed, or may deviate over time in an intentional or unintentional manner. Additionally or optionally, the inflation continuum of the bladder 120 may be expressed as a vertical displacement (e.g. by 1.5 inches) of the bladder 120 as a result of inflation, i.e. movement between a greater inflation percentage and lesser inflation percentage.

The inflatable bladder 120 may be mounted on support frame 112 and extend along the length between first end portion 116 and second end portion 118 of the housing 102. In an exemplary embodiment, the bladder 120 and the outer layer 108 are separate components positionable adjacent each other. Specifically, the bladder 120 may be disposed below the outer layer 108, such that when the bladder 120 moves between the inflated state and the deflated state, the outer layer 108 (or a portion thereof) also moves. More specifically, when the bladder 120 further extends along a length of the outer layer 108, the outer layer 108 moves in response to motion of the bladder 120 between the inflated state and the deflated state. The outer layer 108 moves in response to motion of the bladder 120, such that the outer layer 108 moves uniformly along at least a portion of the length (L) of the outer layer 108. Promoting even or uniform displacement of the outer layer in response to motion of the bladder 120 can avoid causing high pressure points at the outer layer-bladder interface. As shown in FIGS. 5A-5B, the outer layer 108 comprises a surface 144 configured to support at least a torso of baby 200, and the surface 144 moves in response to motion of the bladder 120. In particular, when the bladder 120 moves to the relatively inflated state, surface 144 moves along direction 146 and conversely, when the bladder 120 moves to the relatively deflated state, surface moves along direction 148 and returns to a resting state.

The bladder system also includes a fluid handling system, which may comprise a fluid handler, such as a pump 122, configured to drive fluid into or out of the inflatable bladder 120 to simulate the breathing or respiration motion of the bonded human 202. Fluid as used herein may preferably be air, but is not limited thereto. In operation, the pump 122 moves fluid into the bladder 120 such that it moves to the relatively inflated state and moves fluid out of the bladder 120 such that it moves to the relatively deflated state. The pump 122 may be configured to inflate or deflate the bladder 120 at a fixed volume and at an adjustable rate. In one example, the adjustable rate may have a preferable range of 5 to 30 breaths per minute, or a more preferable range of 10 to 20 breaths per minute. Further, the fluid handling system may comprise the fluid handler, the actuation of which may be preferably performed without producing higher noise levels. A lower or nondisruptive noise level generated by the fluid handling system while in operation is more conducive to promoting an effective recovery and/or development environment for the infant patient. Additionally, the lower or nondisruptive noise level decreases the risk of interference with the heart beat provided by the heartbeat simulation system 106 (discussed further below). In an alternative embodiment, the fluid handling device may comprise bellows that are squeezed pneumatically or mechanically to move the bladder 120 between the inflated and deflated states. The use of bellows may be preferable for quickly moving a fixed volume of fluid into and out of bladder 120. This fixed volume of fluid may have a preferable range of 50 mL to 1500 mL of fluid, or a more preferable range of 200 mL to 600 mL of fluid. It should be understood that the volume of fluid associated with the respiration simulation system 104 may depend on factors, such as the desired level of vertical displacement of the bladder 120 (as affected by size of incubator, for example), respiratory frequency, and I:E (inspiratory phase: expiratory phase) ratio, which may have a preferred range of 1:1 to 1:3.

The fluid supplied to the bladder may be from a controlled volume of fluid having a controlled temperature for ensuring a desired temperature of outer layer 108. Accordingly, the bladder system may further comprise a fluid source container and a temperature controller configured to heat or cool the fluid source. In an air-fed system, cooling the fluid supply may include exchanging air between the controlled volume and ambient air, providing a heat pump that provides gentle heating or cooling, or providing sources of relatively hotter air and relatively cooler air (e.g. produced by the output of a heat pump) for mixing together to achieve the desired temperature. In preferred embodiments, the system is engineered to avoid any possibility that a malfunction of a sensor or the heating/cooling system could take the skin temperature out of a preferred range. Other systems for ensuring a desired skin temperature may also be provided, such as that already provided in the incubator 140 wherein heat transfer between the air temperature of the incubator 140 and the skin of the baby 200 is caused by natural convection.

Still further, when the simulator 100 is positionable within the incubator, one or more of the fluid handling device 122 and a power supply configured to drive the fluid handling device 122, are external to the incubator, as shown in FIGS. 6A-6B. The respiration simulation system 104 may then be activated by the power supply driving the fluid handling device 122, which his connected to the bladder 120 through a hose system 154. In this configuration, a lower height profile of the simulator 100 can be achieved. The lower height profile allows for better positioning within the incubator 140. It also permits the simulator 100 to be useable with other medical equipment. In one non-limiting example, the simulator 100, which can gently restrict movement and/or maintain a specific position of the baby 200, can be used in conjunction with other medical equipment such as an X-ray machine, which typically requires a patient to remain still. Further, this lower height profile discourages unnecessary movement of the baby 200, who may be in a more fragile or vulnerable state, between simulator 100 and other medical equipment. As is known in the art, some incubator arrangements are configured with the ability to place a film or digital X-ray image receiver beneath the baby 200 (e.g. in a drawer disposed in the incubator beneath the surface on which the baby 200 rests) without disturbing the baby 200. For this reason, a preferred design of the simulator 100 may be devoid of any X-ray disruptive materials positioned directly underneath the area of the simulator 100 for receiving the baby 200, to minimize any anomalies in X-ray images procured in this manner due to the presence of the simulator 100.

The term “respiration simulation system” is used herein to refer to all components of any system used for simulating respiration. The system, which system may be modular (i.e. readily severable in whole or in part from the rest of simulator 100), or may be integral to (not readily severable in whole or in part from) the simulator 100.

### Heartbeat Simulation System

Also positioned within the housing 102, the heartbeat simulation system 106 is configured to simulate a heartbeat of the bonded human 202. In an exemplary embodiment, the heartbeat simulation system 106 comprises a speaker, e.g. a subwoofer, which produces both haptic and auditory signals perceptible to the baby 200. As shown in FIG. 4, the heartbeat simulation system 106 is positioned beneath the outer layer 108 and the bladder 120. In particular, the heartbeat simulation system 106 may be placed in a centrally located region of the housing 102. This location is intended to mimic where the baby 200 would hear and/or feel the heartbeat of the bonded human 202, when the baby 200 is resting in a prone position on the chest of the bonded human 202, as would be the case during standard Kangaroo care.

In an exemplary embodiment, the heartbeat simulation system 106 is configured to simulate a heartbeat or a heart rate by delivering a pulsed vibration, or a predetermined series or sequence of pulsed vibrations or other haptic signals. The heartbeat simulation system 106 may additionally or optionally simulate the heartbeat as an auditory signal with little or no haptic component. Thus, the heartbeat simulation system may produce a haptic signal, an auditory signal, or a combination thereof.

### Scent Insert

Positioned on outer layer 108, a scent insert 150 is configured to release a scent of the bonded human 202 for an effective amount of time (i.e. a period of time during which the scent continues to be perceivable by the baby 200). In an exemplary embodiment, the scent insert 150 comprises a patch configured to be dispersed with the scent of the bonded human 202 (achieved when the scent insert 150 is applied to the skin of the bonded human 202). Alternatively, the scent insert 150 may be dispersed with other natural or synthetic scents, including but not limited to scents for triggering relaxation and sleep (e.g. lavender oil, etc.), for triggering an awakened state (e.g. peppermint oil, etc.), or for achieving a therapeutic effect (e.g. an inhalable medicinal vapor). The scent insert 150 may be activated (i.e. configured to release the natural or synthetic scent) based on temperature (e.g. heat), air flow, or pressure applied to outer layer 108 on which the scent insert 150 is disposed (as shown in FIG. 3B), or may be activated based on aspects of an environment in which the simulator 100 is used, such as the incubator 140. It should be understood that location of scent insert 150 as illustrated in FIG. 3B is non-limiting and that scent insert 150 may be placed at other locations defined by simulator 100 (e.g. on other portions of outer layer 108, within housing 102, etc.) or locations within an environment in which simulator 100 is positioned (e.g. within incubator 140). Still further, scent insert 150 may optionally comprise other layers, such as a support layer or a permeable cover.

### Communication Interface

Referring now to FIG. 7, a monitoring system 400 for use with simulator 100 is disclosed. The monitoring system 400 comprises a smart device 130 having a smart device communication interface embedded therein. The monitoring system 400 further includes a simulator 100 comprising a simulator communication interface configured for transmitting and receiving information with at least one external communication interface that is external to the simulator 100. In an exemplary embodiment, the at least one external communication interface is embodied in or communicatively connected to a smart device 130, e.g. smart device communication interface (discussed further below). It should be understood from the description herein that communication between the simulator communication interface and the smart device communication interface may be achieved via wireless or wire-based technology. In the case of a wired format, a physical medium (e.g. wire, cable) is required to transmit and receive data between the simulator communication interface and the smart device communication interface. In the case of a wireless format, the simulator communication interface may comprise a wireless transceiver (e.g. Bluetooth®, WiFi, RFID, Cellular, etc.) configured for wireless communication. Specifically, the wireless transceiver is communicatively connected to the smart device communication interface.

Simulator 100 also includes a processor coupled to the display 124 and the simulator communication interface. The processor is configured to receive, via the simulator communication interface, an input relating to one or more biological parameters of the bonded human 202. The one or more biological parameters may include at least a measured respiration rate and a measured heartrate of the bonded human 202. In an exemplary embodiment, the one or more biological parameters of the bonded human 202 may be measured and transmitted to the simulator 100 via the monitoring system 400 configured for use with the simulator 100. This monitoring system 400 comprises one or more sensors configured to measure one or more biological parameters (e.g. heart rate, respiration, body temperature, etc.) of the bonded human 202. The one or more sensors may be embodied within the smart device 130 (as will be discussed further below). The one or more sensors may comprise a heart rate sensor configured to measure pulse waves, which represent changes in volume of a blood vessel as the heart pumps blood. Additionally or optionally, the heart rate sensor may be configured to provide a respiration rate based on the measured heart rate (i.e. the time between heart beats decreases when the bonded human 202 inhales and increases when the bonded human 202 exhales). Still further, the one or more sensors may include temperature sensors configured to measure skin temperature of the bonded human 202. These biological parameters (e.g. heart rate, respiration, temperature, etc.) are important for simulating Kangaroo care, because via direct skin-to-skin contact between the baby 200 and the bonded human 202, the heart rate, respiration rate, and skin temperature of the bonded human 202 can be perceived (felt or heard) by the baby 200.

As shown in FIG. 7, to facilitate the measurement and/or monitoring of the one or more biological parameters of the bonded human 202, the smart device 130 may comprise wearable technology. Wearable technology can include fitness trackers in the form of wristbands or straps, such as Fitbit®. Wearable technology can also include smart jewelry (e.g. rings, watches and pins) and smart clothing, all of which may be configured for fitness or health monitoring via use of one or more sensors embodied therein. The wearable smart device 130 may include a local processor that is programmed with machine readable instructions for causing the one or more sensors to monitor and detect the relevant measurements, i.e. for the heart rate sensor to monitor and detect the heart rate and the respiration rate of the bonded human 202, and for the temperature sensor to monitor and detect skin temperature. Additionally, the local processor of the wearable smart device 130 may be programmed with machine readable instructions for causing the one or more sensors to transmit a signal corresponding to the one or more biological measurements from the communication interface connected to the smart device 130 via a global communication network 156 to another communication interface connected to the human simulator 100. The global communication network 156 may include a second processor for interfacing the communication interface connected to the smart device 130 and the another communication interface connected to the human simulator 100.

In an alternative embodiment, smart device 130 may be configured to directly connect to the global communication network 156 or connects (via Bluetooth®) to an application on a mobile device (e.g. phone). In this configuration, the smart device 130 comprising the mobile device includes the local processor that is programmed with machine readable instructions for causing the one or more sensors to monitor and detect the relevant measurements (as discussed above). Further, the local processor on the mobile device is also programmed with machine readable instructions for causing the one or more sensors to transmit a signal corresponding to the one or more biological measurements from the communication interface connected to the smart device 130 via the global communication network to another communication interface connected to the human simulator 100.

Based on the received input, such as the measured respiration rate of the bonded human 202, the processor of the simulator 100 is configured to activate the respiration simulation system 104. In particular, the processor is configured to control the rate of inflation and deflation of the bladder 120, such that the bladder 120 moves between the relatively inflated state and the relatively deflated state at a rate that corresponds to the measured respiration rate of the bonded human 202. This is consistent with the benefits provided by direct skin to skin contact in standard Kangaroo care, because the simulator 100 is configured to mimic the interaction between the baby 200 and the bonded human 202, which includes the baby being able to perceive (e.g. feel or hear) the respiration of the bonded human 202. In another exemplary embodiment, the processor is configured to activate the respiration simulation system 104 based on the bonded human’s measured respiration volume, such as determined through plethysmography signals, which may be embodied in the smart device 130 worn by or adjacent the bonded human 202.

Likewise, the processor is configured to activate the heartbeat simulation system 106 based on the received input, such as the measured heart rate of the bonded human 202. In this way, the processor is configured to control the pulsed vibration, or a predetermined series or sequence of pulsed vibrations, provided by the speaker 106, such that the pulsed vibrations may correspond to the measured heart rate of the bonded human 202. This is also consistent with the benefits provided by direct skin-to-skin contact in standard Kangaroo care, because the simulator 100 is configured to mimic the interaction between the baby 200 and the bonded human 202, which includes the baby being able to perceive (e.g. feel or hear) the heart rate of the bonded human 202.

However, in order to avoid simulation of a sudden and rapid onset of respiration rate (or breathing frequency) and/or heart rate by the simulator 100, because the bonded human 202 may experience tachypnea or tachycardia during the course of the simulation, the processor of the simulator 100 may be configured to include a programmable setting in which when the detected biological parameter (e.g. heart rate or respiration rate) exceeds a predetermined threshold, then the simulator 100 is configured to operate under default conditions. The default conditions may include activating the respiration simulation system 104 within a predetermined range of inflation/deflation rate of the bladder 120 and/or activating the heartrate simulation system 106 within a predetermined range of sound / haptic amplitude and frequency. Likewise, if the bonded human 202 is unavailable for monitoring (e.g. away from smart device 130 or a distance apart from the baby 200), or to reduce complexity, the processor may be configured to cause the respiration simulation system 104 and/or the heartbeat simulation system 106 to operate under default conditions. Setting default conditions can help ensure that the respiration rate and/or heart rate do not exceed a predetermined safe limit for the infant patient. Further, the processor may be configured to include a programmable setting in which when the simulator 100 is operating under default conditions and/or a loss of connection between the smart device 130 and the simulator 100 occurs, the default conditions may be adjusted in an override mode (after entry of appropriate credentials by hospital staff, for example).

Additionally, it should be understood that some embodiments may be programmed to provide default conditions at all times without real-time or near-time monitoring of a specific human. The default conditions may be set to a specific rhythm of respiration and heart rate that is fixed or variable within a range. Variable rhythms may be preferred in some embodiments to better mimic actual human rhythms. Exemplary “ideal” rhythms may be captured and saved over a period of time from the actual human activity of a test subject (e.g. not a specific bonded human 202 with a specific relationship to a specific baby), and may include rhythm patterns for sleep, activity, and exercise, which rhythms may be user-selectable or automatically cycled in any order in a regular or irregular fashion. Other exemplary rhythms may include music, soundscapes (e.g. “humming” sounds), white noise, prerecorded messages, songs, and/or sounds from the bonded human 202, and a combination thereof. The rhythms may be further adjusted based on frequency modes (e.g. a cat purring sound at 25-150 Hz).

Still further, the processor is configured to receive, via the simulator communication interface, information relating to operation of the simulator 100 and/or information relating to one or more biological parameters of the baby 200, when the baby 200 is in contact with the outer layer 108. The one or more biological parameters of the baby 200 may be detected similarly as described above with respect to the bonded human 202, i.e. via use of one or more sensors or via use of other medical equipment in the vicinity of the simulator 100 (such as equipment already provided for use with the incubator 140). Monitoring of the baby in conjunction with the respiration and heart rate stimulus provided by the simulator 100 may show favorable or unfavorable responses to the stimulus, and may inform the use of specific real or simulated rhythms for specific purposes or for specific infants with specific indications.

Simulator 100 may further comprise a display 124 mounted on the housing 102, such as on support frame 112 as shown in FIG. 1A or on one of the first end portion 116 or the second end portion 118 as shown in FIG. 7. The display 124 is connected to the processor and is configured to display information relating to operation of the simulator 100. Additionally or optionally, the display 124 is configured information relating to the one or more biological parameters of the baby 200, the bonded human 202, or a combination thereof. The display 124 may comprise a capacitive or pressure touch screen user interface that is integrated into the display 124. The pressure touch screen user interface may be preferable in a sterile or clinical environment, where medical professionals and the bonded human 202 may be required to wear gloves. The capacitive screen user interface may be preferable in an environment wherein gloves are not specifically required, e.g. home environment.

Although monitoring system 400 is illustrated in FIG. 7, with simulator 100 being positioned within incubator 140, it should be understood that monitoring system 300 may be applied in other environments, such as within the hospital (i.e. without incubator 140) or the home.

### Sling/Home Embodiment

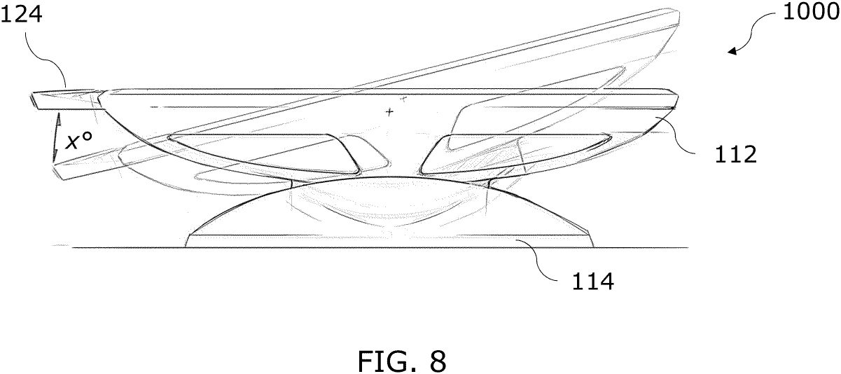

Another embodiment of the simulator 100 made according to the present invention is illustrated in FIG. 8. The components of this second embodiment, such as simulator 1000, generally correspond to those of the first embodiment. However, in this second embodiment, the simulator 1000 differs in that the housing 102 is pivotable relative to the support frame 112 and/or base 114 about a pivot axis. In particular, the housing 102 is pivotable between a first position and a second position. When the housing 102 is in the first position, the housing 102 is parallel to the base 114. When the housing 102 is in the second position, the housing is oriented obliquely at an angle of x° relative to the base 114, wherein x is preferably in a range of 0 to 75 degrees, and more preferably in a range of 0 to 45 degrees.

In addition, simulator 1000 is suited for use in a non-clinical environment, such as for residential home use, such as when the baby 200 and the bonded human 202 are out of the hospital. In this embodiment, the housing 102 is pivotable to allow the baby 200 to perceive the surrounding environment, such as the home.

### Method

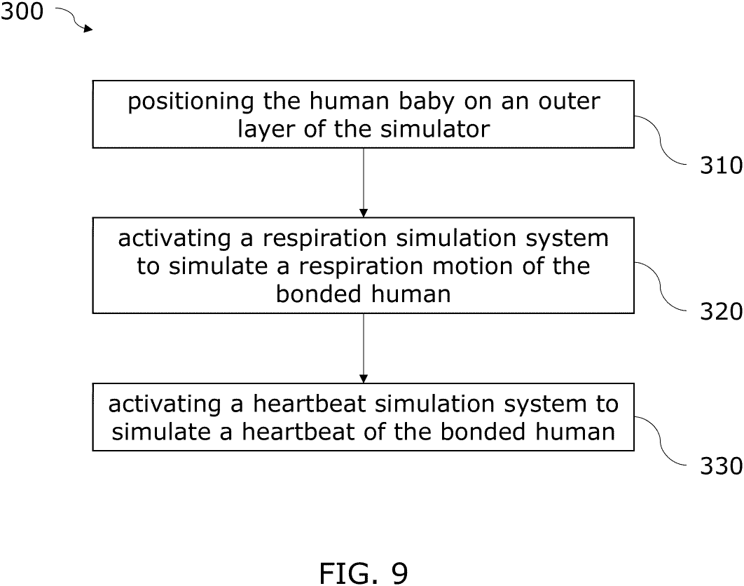

Referring now to FIG. 9, a method of using the simulator 100 or simulator 1000 is provided. The method 300 includes one or more steps including positioning the human baby on an outer layer of the simulator; activating a respiration simulator to simulate a respiration motion of the bonded human 202; and activating a heartbeat simulator to simulate a heartbeat of the bonded human 202. Additional details of method 300 are set forth below with respect to the elements of simulator 100, but it should be understood that method 300 is additionally applicable for use with simulator 1000.

In step 310, the human baby is positioned on an outer layer of the simulator. In an example, the human baby 200 is positioned on the outer layer 108 of the simulator 100. The outer layer 108 comprises biocompatible material selected to have one or more properties configured to mimic human skin (e.g. silicone). The outer layer 108 may include a surface that is contoured to mimic a torso, womb, or chest of a bonded human 202.

In step 320, a respiration simulation system to simulate a respiration motion of the bonded human is activated. In an example, the respiration simulation system 104 comprises the bladder 120 configured to move between the inflated state and the deflated state and the fluid handling device configured to drive the bladder. Fluid handling device may comprise pump 122. The pump 122 moves the bladder 120 between the inflated state and the deflated state at a predetermined respiration rate. Additionally or optionally, the respiration step 320 includes detecting, via one or more sensors disposed adjacent the bonded human 202, a respiration rate of the bonded human 202, and the pump 122 is activated to move the bladder 120 between the inflated state and the deflated state based on the detected respiration rate of the bonded human 202.

In step 330, a heartbeat simulation system to simulate a heart rate of the bonded human is activated. In an example the heartbeat simulation system 106 is activated at a predetermined heart rate. Additionally or optionally, the heartbeat step 330 includes detecting, via one or more sensors disposed adjacent the bonded human 202, a heart rate of the bonded human 202, and the heartbeat simulation system 106 is activated to simulate a heart rate based on the detected heart rate of the bonded human 202.

In an optional step, the method 300 further comprises the steps of detecting, via one or more sensors, one or more biological parameters (e.g. heart rate, respiration rate, body temperature, etc.) of at least one of the baby 200 and the bonded human 202. Simulator 100 may comprise display 124 that is configured for displaying information relating to the one or more biological parameters of the baby 200 and the bonded human 202.

As used herein, one or more features may be described as “based on” another parameter (e.g. the heartrate of the heartbeat simulation system may be based on the actual heart rate of the bonded human 202). It should be understood that as used herein, the term “based on” is not intended to limit the basis for the feature only to the parameter noted, and that the feature may be based only in part on the noted parameter as well as in part on one or more other parameters or control factors, which may be expressly discussed or may not be expressly discussed (i.e. the heartrate may be based on the actual heart rate of the bonded human 202 but also may be based on (e.g. confined by) a limitation range).

It should be understood that any of the algorithms as described herein may be embodied on computer readable media readable by an associated processor for causing the simulator 100, portion thereof, or other components described herein to operate as described.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A human simulator configured to simulate interaction between a human baby and a bonded human, the simulator comprising:
a housing having an outer layer configured to contact the baby, the housing having a surface contoured to simulate a torso of the bonded human;
a respiration simulation system positioned at least in part within the housing and configured to simulate a respiration motion of the bonded human;
a heartbeat simulation system positioned at least in part within the housing and configured to simulate a heartbeat of the bonded human;
wherein the respiration simulation system comprises a bladder system comprising an inflatable bladder having a relatively inflated state and a relatively deflated state, and a fluid handling system configured to move fluid into or out of the inflatable bladder between the relatively inflated state and relatively deflated state to simulate the respiration motion; and
wherein the fluid handling system comprises a fluid handler for causing transport of the fluid in and out of the bladder, a power supply for operating the fluid handler, and a temperature control system for controlling a temperature of the fluid transported by the fluid handling system.

2. The human simulator of claim 1, wherein the human simulator further comprises:
a first communication interface configured for transmitting and receiving information with at least one second communication interface external to the simulator, first communication interface including a wireless transceiver;
a processor coupled to the first communication interface, the processor is configured to:
receive, via the first communication interface, information relating to operation of the human simulator and information relating to one or more biological parameters of the baby in contact with the outer layer;
receive, via the first communication interface, an input relating to one or more biological parameters of the bonded human, including at least a measured respiration rate and a measured heartrate;
activate the respiration simulation system based on the received input, including activating the respiration simulation system in a timed sequence in conformance with the measured respiration rate of the bonded human; and
activate the heartbeat simulation system based on the received input, including activating the heartbeat simulation system in a timed sequence in conformance with the measured heart rate of the bonded human; and
a display connected to the processor and configured to display information relating to operation of the human simulator and information relating to the one or more biological parameters of the baby, the bonded human, or a combination thereof.

3. (canceled)

4. The human simulator of claim 1, wherein the outer layer comprises material having one or more properties selected to simulate human skin, the outer layer further comprising an anti-microbial material.

5. (canceled)

6. The human simulator of claim 1, wherein the housing extends along a length between a first end portion and a second end portion, the contoured outer layer of the housing including a concave area for receiving a head portion of the human baby, and wherein the contoured outer layer further comprises a pillow for providing support to the head portion and a neck portion of the human baby.

7. (canceled)

8. (canceled)

9. The human simulator of claim 2, wherein the wireless transceiver is communicatively connected to the at least one second communication interface, wherein the at least one second communication interface is embodied in or communicatively connected to a smart device configured to measure and transmit the one or more biological parameters of the bonded human to the human simulator.

10. (canceled)

11. The human simulator of claim 9, wherein the one or more biological parameters are detected by one or more sensors embodied in the smart device, the smart device configured to communicatively connect to a global communication network or to a mobile device.

12. (canceled)

13. (canceled)

14. (canceled)

15. (canceled)

16. (canceled)

17. (canceled)

18. (canceled)

19. The human simulator of claim 1, wherein the bladder and outer layer are separate components positioned adjacent each other, with the bladder extending along a length of the outer layer.

20. (canceled)

21. (canceled)

22. The human simulator of claim 1, wherein the human simulator is positionable within an incubator, and at least portions of the fluid handling system, are disposed external to the incubator.

23. (canceled)

24. (canceled)

25. The human simulator of claim 1, wherein the heartbeat simulation system is configured to provide a simulated heart rate as a pulsed vibration, a predetermined series or sequence of pulsed vibrations, an auditory signal, or a combination thereof.

26. (canceled)

27. The human simulator of claim 3, wherein the display comprises a capacitive or pressure touch screen user interface integrated into the display.

28. The human simulator of claim 1, wherein the housing is mounted on a base configured to support at least the housing, such that the housing is elevated relative to the base, and wherein the housing is pivotable about a pivot axis between a first position and a second position, wherein in the first position, the housing is parallel to the base and in the second position, the housing is oriented obliquely relative to the base.

29. (canceled)

30. A method of using a human simulator configured to simulate interaction between a human baby and a bonded human, the steps comprising:
positioning the human baby on an outer layer of the human simulator;
activating a respiration simulation system to simulate a respiration motion of the bonded human via activating a fluid handling system to transport fluid into and out of an inflatable bladder to move the bladder between a relatively inflated state and a relatively deflated state at a predetermined respiration rate;
activating a heartbeat simulation system to simulate a heartbeat of the bonded human via detecting, via one or more sensors disposed adjacent the bonded human, a heart rate of the bonded human, and activating the heartbeat simulation system to simulate a heart rate based on the detected heart rate of the bonded human; and
wherein the respiration step further includes detecting, via one or more sensors disposed adjacent the bonded human, a body temperature of the bonded human, and activating a temperature controller for the fluid handling system to control a temperature of the fluid based on the detected body temperature of the bonded human.

31. (canceled)

32. (canceled)

33. (canceled)

34. (canceled)

35. (canceled)

36. The method of claim 30, wherein the heartbeat step includes detecting, via one or more sensors disposed adjacent the bonded human, a heart rate of the bonded human, and activating the heartbeat simulation system to simulate a heart rate based on the detected heart rate of the bonded human.

37. The method of claim 30, further comprising detecting, via one or more sensors, one or more biological parameters of at least one of the baby and the bonded human, and displaying information relating to the one or more biological parameters of the baby and the bonded human.

38. A monitoring system for use with a human simulator configured to simulate interaction between a human baby and a bonded human, the monitoring system comprising:
a smart device having a first communication interface;
one or more sensors configured to detect one or more biological parameters of the bonded human;
at least one processor;
wherein the at least one processor is programmed with machine readable instructions to activate the one or more sensors and to transmit a signal corresponding to the detected biological parameters of the bonded human from the first communication interface via a global communication network to a second communication interface connected to the human simulator.

39. The monitoring system of claim 38, wherein the smart device comprises wearable technology, and wherein the first communication interface is a wireless transceiver configured to connect to the global communication network, a mobile device, or a combination thereof.

40. (canceled)

41. The monitoring system of claim 39, wherein the at least one processor includes a local processor embodied in the smart device, the local processor being connected to the first communication interface and the one or more sensors.

42. The monitoring system of claim 41, wherein the at least one processor includes an external processor for interfacing the first communication interface connected to the smart device and a second communication interface connected to the human simulator.

43. The monitoring system of claim 39, wherein the mobile device comprises a computer memory medium programmed with instructions for storing and analyzing the detected biological parameters of the bonded human.

44. A non-transitory computer memory medium programmed with machine readable instructions for operating the human simulator of claim 1, the machine readable instructions comprising instructions for causing the heartbeat simulation system and the respiration simulation system to be activated in accordance with an algorithm or program stored in the computer memory medium, and wherein the bonded human is a simulated human, and the algorithm or program stored in the computer medium is programmed with information compiled from information corresponding to a plurality of living humans.

45. (canceled)

46. (canceled)

47. (canceled)
